(19) United States
(12) Patent Application Publication
(10) Pub. No.: US 2016/0213624 A1
(43) Pub. Date: Jul. 28, 2016

(54) Composition, Commericial Product and Method for Treating Cannabis Toxicity

(71) Applicant: Tom Lindeman, Bellevue, WA (US)

(72) Inventor: Tom Lindeman, Bellevue, WA (US)

(21) Appl. No.: 15/005,932

(22) Filed: Jan. 25, 2016

Related U.S. Application Data
(60) Provisional application No. 62/107,248, filed on Jan. 23, 2015.

(51) Int. Cl. A61K 31/05; A61K 31/015; A61K 9/00; A61K 36/9068; A61K 31/714; A61K 9/107; A61K 31/7068; A61K 36/185

(52) U.S. Cl. CPC A61K 31/05; A61K 31/7068; A61K 31/015; A61K 36/185; A61K 36/9068; A61K 31/714; A61K 9/107; A61K 9/0053

(57) ABSTRACT

The present invention provides a novel emulsion composition in a stable, fast acting emulsion that contains cannabidiol in an aqueous vehicle. The cannibidiol is extracted using CO2 from an industrial hemp plant that produces cannibiodiol molecules surrounded by less fats and waxes making it more hydrophilic. A little emulsifying agent, such as polysorbate 80, is mixed with cannabidiol oil to create a stable first emulsion mixture. A batch mixture is then prepared containing water, food preservatives, sugar, a sufficient amount of CBD Premix that contains citicoline and masker A for Reb A. Both mixtures are then mixed in water with ginger, Vitamin C, Vitamin B6, Vitamin B12, and sugar. The final mixture is then distributed in 60 ml bottles and consumers are instructed to take an initial loading dose of 15 ml (equal to approximately 5 mg of cannabidiol) either before a THC containing product is in consumed or smoke, shortly after a THC containing consuming or smoking or when the first signs of anxiety are perceived.

This utility patent application is based on and claims the filing date benefit of U.S. Provisional Patent Application (Application No. 62/107,248) filed on Jan. 23, 2015.

Notice is given that these patent document contains original material subject to copyright protection. The copyright owner has no objection to the facsimile or digital download reproduction of all or part of the patent document, but otherwise reserves all copyrights.

### BACKGROUND OF THE INVENTION

1. Field of the Invention

This invention pertains to compositions, commercial packages and methods for treating symptoms of cannabis toxicity.

2. Description of the Related Art

Cannabis legalization is spreading through the U.S. at a fast pace, and cannabis use is growing, with millions of American using cannabis each day. According to MMJ Business Daily the U.S. cannabis market outlook is $3B in 2015 and growing to $8B in 2018 (http://mmjbusinessdaily.com/new-forecast-u-s-medical-marijuana-and-recreational-cannabis-sales-to-hit-8-billion-by-2018/). Currently, four states allow recreational cannabis, and twenty five states allow medical cannabis. In Washington State alone, there are over 400 medical marijuana dispensaries.

Although impossible to have a lethal toxic overdose of cannabis, it is possible to consume more than necessary leading to unwanted effects of a potentially severe nature. Because individuals have different physical characteristics or may have other food products in their system, this can affect how cannabis is absorbed. The fact that the stomach doesn't absorb marijuana evenly or predictably when taken in oral form, it can be harder for people to estimate how much they need to eat to get the desired effects. There is also a significant delay in the time (e.g. 60 to 90 minutes) for cannabis to have its effect. This can cause people to think they haven't eaten enough marijuana, so they continue to eat more, leading to an overdose situation.

THC (Tetrahydrocannabiol) is the main mind-altering chemical found in the cannabis plant. When someone smokes too much marijuana or eats too much edibles infused with cannabis, they may experience severe anxiety, ataxia/dizziness, sweating, nausea, vomiting, and a sensation of dying. Additional symptoms of marijuana overdose may include disorientation, feeling delirious or feverish, and can be followed by malaise or stupor commonly associated with a ‘hangover’. In some severe cases, individuals who overdose may also become extremely paranoid, hallucinate, or experience panic attacks making them harmful to themselves or others.

Fortunately, ingesting of excessive cannabis is not considered fatal nor cause organ damage. Usually the best treatment is to sleep.

Cannabidiol, (also known as ‘CBD’), is one of at least 111 cannabinoids identified in cannabis and in industrial hemp. Cannabidiol is regarded as non-psychoactive and blocks THC.

The FDA of the United States considers hemp oil (and its derivative CBD) to be a dietary supplement (not a medication), since they are made from industrial hemp plants. This means that in the United States, a medical prescription is not needed and anyone can legally purchase and consume industrial hemp products in every state.

Cannabis may be inhaled (smoking or vaporization), ingested (eating or drinking or absorption through the mucous membranes of the mouth), or applied topically to the skin. Inhalation is considered more rapid and a more efficient delivery method than ingestion because the cannabinoids, terpenes, and other chemicals readily pass through the lungs and directly into the bloodstream. When ingested, the THC and cannabinoids are slowly absorbed in the stomach and then travel to the liver where they undergo oxidation. The oxidation and the concentration of the oxidized products produce can vary. Larger amounts of cannabis must be taken if ingested, and the effects can vary and last longer and cause toxicity symptoms.

What is needed is a fast acting, room temperature stored, oral composition in an emulsion containing a specific amount of cannabiodiol per dose extracted from a legalized source, such as industrial hemp, that counteracts the psychological and physiological toxic effects of THC.

### SUMMARY OF THE INVENTION

The present invention relates to using a formula comprising a combination of natural herbs, plant extracts, vitamins and minerals used to help alleviate toxicity symptoms and risks related to negative effects due to the overuse of cannabis or cannabis containing products. In one embodiment, the composition is a single dose (60 mls) stored in a package with printed instructions.

More specifically, the composition is an emulsion comprises 10 to 50 mg of cannabidiol, 200 to 500 mg of citicoline, 10 to 50 mcg of a-pinene, 500 to 1000 mcg of ginger, 100 to 250 mg of Vitamin C, 10 to 50 mg of Vitamin B6, and 50 to 200 mcg of Vitamin 12. The above ingredients are mixed in suitable aqueous vehicle to produce a final 60 ml volume. Polysorbate 80 is a surfactant/emulsifier. Berry juice, sugar or artificial sweetener (1 to 6 mgs), guayusa and artificial coloring may be added. Potassium sorbate and sodium benzoate may be added as food preservatives.

In an alternative embodiment, food grade glycerin and propylene glycol may be added that enables the composition to be heated and vaporized in an electronic vaporizing device.

In another embodiment, the above ingredients may also be mixed and formulated in tablet or capsule.

In another embodiment, the above ingredients may also be mixed and formulated into an oral spray.

In another embodiment, the above ingredients may be mixed and formulated into a chewable gum.

In another embodiment, the above ingredients may be mixed and formulated into a sublingual film or lozenge.

Upon onset of toxicity symptoms, the entire composition is consumed.

### DESCRIPTION OF THE DRAWINGS

FIG. 1 is a front elevational view of a display box with a 12 commercial packages containing the composition used to treat cannabis toxicity symptoms.

FIG. 2 is side perspective view of the display box in FIG. 1.

FIG. 3 is an illustration showing the intensity of the physiological and psychological effects of THC in an individual over time and the composition may be taken and showing on the duration of the effects are reduced.

FIG. 4 is a front plan view of the container.

FIG. 5 is a plan of the label.

FIG. 6 is a table showing the ingredients in the composition.

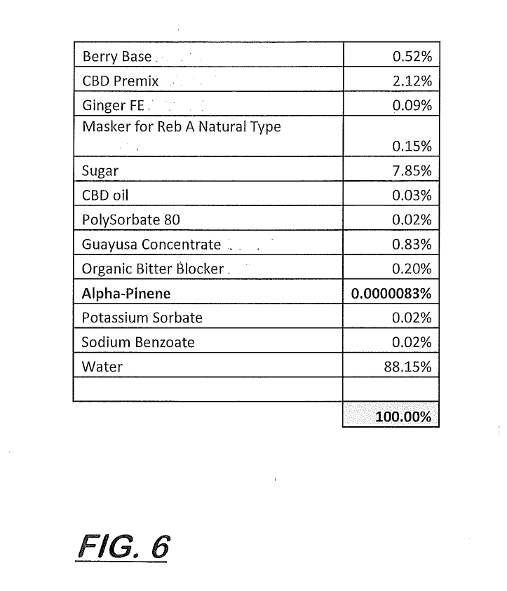

### DESCRIPTION OF THE PREFERRED EMBODIMENT(S)

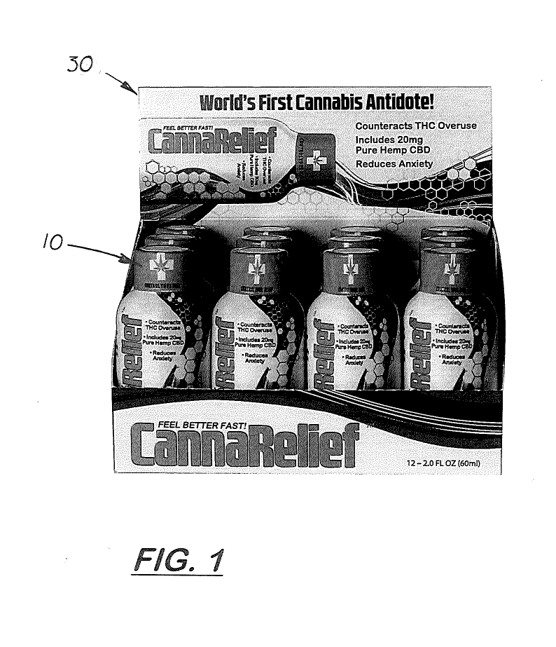

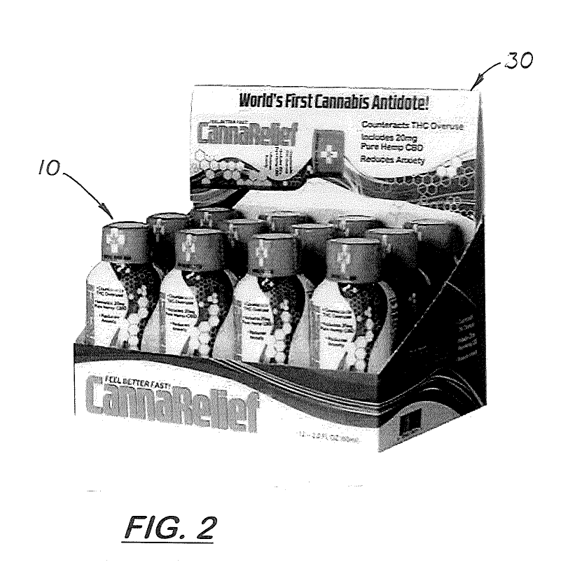

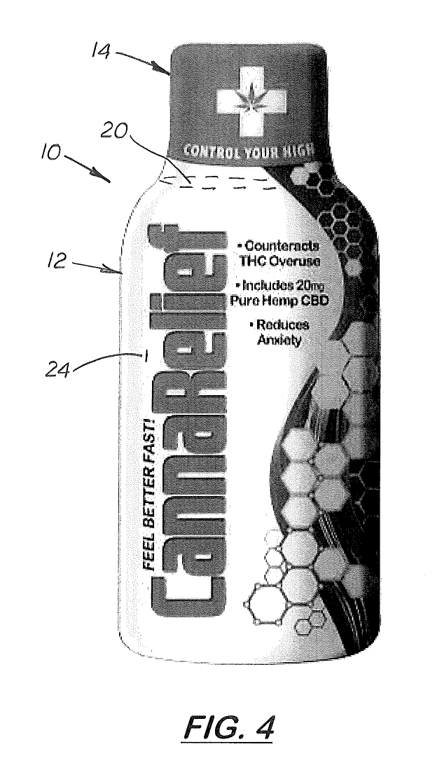

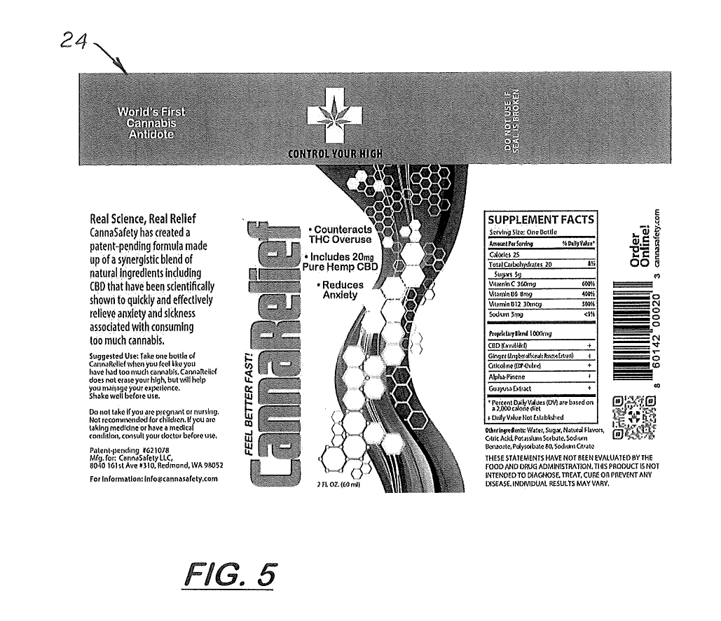

Disclosed is a stable, liquid composition used to treat anticipated or ongoing symptoms of cannabis overuse or overdose. The liquid composition 20 is sold in 2 fl. oz containers designed to be consumed entirely as a single 2 fl oz dose or one or two 1 fl. ounce doses depending on the user's anticipated or actual symptoms. FIGS. 1, 2 and 4 show the composition 20 being dispensed in an over the container 12 with a sealed cap and an outer label 24 with the main ingredient (CBC) and the amount contained inside the container 12. The outer label 24 shown more clearly in FIG. 5 lists the main ingredients in the composition 20. FIG. 1 is a front elevational view of a display box with a 12 commercial packages each containing 2 fl. oz. of the composition 20 advertising its use to treat cannabis toxicity symptoms. FIG. 2 is side perspective view of the display box in FIG. 1.

The liquid composition 20 is a two phase emulsion combining two immiscible liquids, —CBD (cannabidiol) and water. A first emulsion mixture is made by first manufacturing a starting emulsion mixture made from CBD resin containing approximately 25% CBD produced using a CO2 based extraction method. The CBD resin is mixed with a surfactant/emulsifier, such as Polysorbate 80. The CBD resin and Polysorbate 80 mixture is then added to approximately 25% of the final volume of water needed to make a known total volume of the composition. In the preferred embodiment, the CBD resin/polysorbate 80 mixture and water are mixed in a high sheer mixer (i.e. Silverson Mixer Homogenizer). A small amount (0.00001 to 0.001%) A-pinene may be added to the first emulsion mixture.

Next, the batch mixture is preparing comprising 60 to 70% of the original volume of water. Food preservatives, Potassium Sorbate and Sodium Benzoate is then added and mixed for approximately 5 minutes. A little sucrose is then added. Next, a proprietary product sold under the trademark CBD Premix by Allen Flavors, Inc. in Edison, N.J. is then added to the mixture. The CBD Premix contains Vitamin C, Vitamine B6, Vitamin B12, and Citicoline. Next, a nature sucrose enhancer and a masker for Reb A are added and mixed.

After the batch mixture is manufactured, both the first emulsion mixture and the batch mixture are mixed in a large mixer. Next, Guayusa Concentrate is then added and mixed. Ginger, Organic Bitter Blocker and Organic Masking Agent are then added. Next, a berry base is added. The remaining water is then added and the entire composition is mixed for 20 minutes.

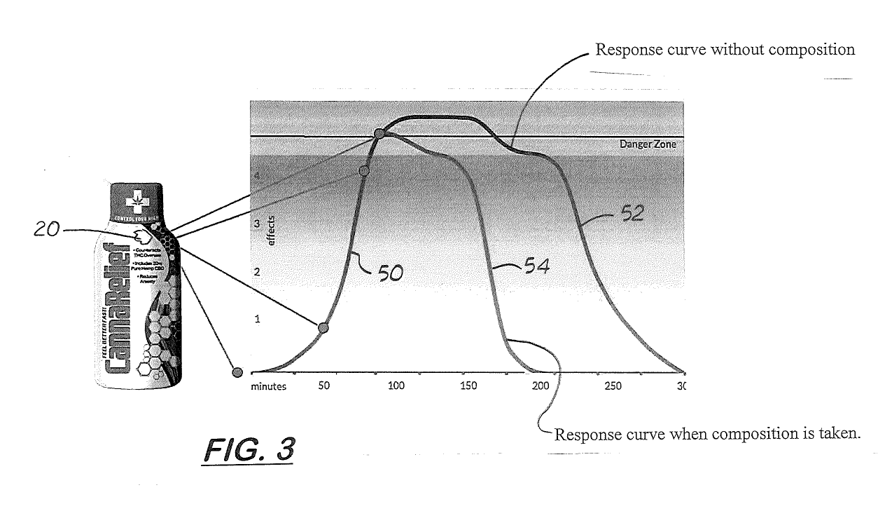

When anticipating or experiencing negative or toxicity symptoms associated with cannabis overuse/overdose, all or a portion of the composition is orally consumed. Such symptoms may include dizziness, nausea, headache, physical sickness, vomiting, loss of balance, blurred vision, panic, anxiety and mental confusion. As shown in FIG. 3, all or some of the composition 20 may be taken prior to consuming cannabis, shortly after taking cannabis as the symptoms are experienced, after taking cannabis but before the symptoms of toxicity are experienced, or after taking cannabis when the symptoms of toxicity are experienced. When the composition 20 is consumed, the duration of the effects of cannabis are substantially reduced.

In the embodiment described herein, the CBD resin is derived from industrial hemp and contains approximately 25% CBD. In the preferred embodiment, the CBD resin is produced from industrial hemp using CO2 extraction process which produces less foul taste and cleaner product with less plant material. In the preferred embodiment, 2 fl. oz of the composition contains approximately 20 mg of CBD. It should be understood that the CBD (20 mg) in the composition and its potency 20 mg/2 fl. oz) may also vary. The important feature is the source of CBD is from legal source (industrial hemp) and produced by a CO2 extraction processes that produces less foul aftertaste and less plant material.

The citicoline (200-500 mg) is a water-soluble compound essential for the synthesis of phosphatidyl choline, a constituent of brain tissue. It is postulated that citicoline plays a role in neurotransmission and can help support brain function and has been shown to reduce psychoactive effects of THC.

A-pinene, (also known as ‘essential pine oil’) (10-50 mcg) is a terpene extracted from black peppercorns or pine nuts which can decrease anxiety associated with marijuana use. A-pinene is the most common terpene in the plant world and one often found in cannabis, is a bronchodilator potentially helpful for asthmatics. A-pinene also promotes alertness and memory retention by inhibiting the metabolic breakdown of acetylcholinesterase, a neurotransmitter in the brain that stimulates these cognitive effects.

Guayusa is a natural herb that contains theobromine and L-theanine and has stimulatory effects similar to tea and coffee.

Ginger (500-1000 mg) as an extract which reduces nausea associated with cannabis use. Vitamin C is an antioxidant. In one embodiment, the dosage is between 500 mg and 1,600 mg.

Vitamin B6 is found in an array of foods including fortified cereals, beans, meat, poultry, fish, fruits and vegetables and considered a nervous system modulator. In one embodiment, the dosage of Vitamin B6 is between 10 to 60 mg.

Vitamin B12 is postulated to be a nervous system modulator. In one embodiment, the dosage includes between 40 mcg and 80 mcg.

Sugar increases blood sugar levels and help reduce dizziness and nausea.

Sodium Benzoate is a preservative to extend product shelf life.

In yet other embodiments, the supplement can further include:

Artificial coloring;

Quinine or quinine alkaloids (200-350 mg) that prevent or relax muscle trauma or convulsion;

Vitamin E or D-Alpha Tocopheryl Acetate (15 IU to 60 IU, respectively) which is an antioxidant;

Niacin (100 mg and 220 mg); and,

Folic Acid (Vitamin B9).

Caffeine, Kava, and L-theonine

Flavoring and masking ingredients.

The above composition is distributed in a rigid package that holds at least 60 mls of composition. Printed on a panel attached to the package or on an insert is the following instructions:

“RECOMMENDED USE: Take one full bottle for relief of discomfort due to overuse of cannabis. A second bottle may be taken after 30 minutes if symptoms do not decrease. Do not exceed 3 bottles in a 12 hour period.”

I claim:

1. A method of treating undesirable or overdose symptoms associated with consumption of excessive cannabis comprising is an aqueous emulsion containing 10 to 50 mg of cannabidiol oil, citicoline, a-pinene, ginger, guayusa, polysorbate 80, and potassium sorbate, and artificial or natural sweeteners.

2. The method as recited in claim 1, wherein the composition further includes sodium benzoate.

3. The method as recited in claim 1, wherein the composition further includes potassium sodium.

4. The method as recited in claim 2, wherein the composition further includes potassium sodium.

5. The method as recited in claim 1, wherein the composition is consumed prior to consuming cannabis.

6. The method as recited in claim 1, wherein said composition is consumed after consuming cannabis

7. A liquid composition for treatment of cannabis toxicity, 2 fl oz of said composition includes the following ingredients and amount;
a. CBD 10-50 mg derived from industrial hemp extracted using CO2;
b. citicoline 200 to 500 mg;
c. a-pinene 10 to 50 mcg.;
d. polysorbate 80 0.01 to 0.02%; (v/v)
e. guayusa 5 to 2%;
f. ginger 500 mcg to 1000 mcg;
g. a sweetener 1 to 8%; (v/v)
h. at least one preservative taken from a group that includes sodium sorbate and sodium benzoate; and,
i. a sufficient amount of water to produce final volume of 2 fl. oz.

8. The liquid composition as recited in claim 7, further including 100 to 500 mg of Vitamin C.

9. The liquid composition as recited in claim 7, further including 10 to 500 mg of Vitamin B6.

10. The liquid composition as recited in claim 7, further including 50 to 200 mcg of Vitamin B12.

11. A method for manufacturing an oral liquid composition containing 20 mg of CBD in 2 fl. oz; comprising the steps of:
a. preparing a first emulsion mixture made from CBD resin from an industrial hemp source containing approximately 25% CBD produced using a CO2 based extraction method and mixing a sufficient amount of CBD resin to produce a composition containing 20 mg of CBD in 2 fl. oz, adding a surfactant/emulsifier and then adding the emulsion mixture to small volume of water and then mixing the emulsion mixture and water and adding sufficient amount of A-pinene to the emulsion mixture to produce a final concentration of approximately 0.0000083%;
b. preparing a batch mixture containing water, food preservatives, sugar, a sufficient amount of CBD Premix to provide 200 to 500 mg of citicoline 200 to 500 mg per 2 fl. oz, and masker A for Reb A; and,
c. mixing the first emulsion mixture with the batch mixture;
d. adding guayusa ginger, bitter blocker and masking agent and berry base; and,
e. adding additional water to make 2 fl. oz of liquid composition.
